SEQUENCE LISTING (3 sequences)

SEQ ID NO: 1 - protein, 736 aa, Artificial Sequence
  synthetic
  MISC_FEATURE (470)..(470): wherein X can be Ser or Asn
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDTESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSXMASQARNWVPGPSYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

SEQ ID NO: 2 - DNA, 3695 bp, Artificial Sequence
  synthetic
ctctttgcgcttgcgttttcccttgtccagatagcccagtagctgacattcatccggggtcagcaccgtttctgcggactggctttctacgtgaaaaggatctaggtgaagatcctttttgataatctcatgcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggagactttatctgacagcagacgtgcactggccagggggatcaccatccgtcgccccggcgtgtcaataatatcactctgtacatccacaaacagacgataacggctctctcttttataggtgtaaaccttaaactgccgtacgtataggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgaattgtaatacgactcactatagggcgaattgaatttagcggccgcgaattcgcccttatgatatctggtgggaggagggcaagatgacggccaaggtcgtggagtccgccaaggccattctcggcggcagcaaagtgcgcgtggaccaaaagtgcaagtcgtccgcccagatcgatcccactcccgtcattgtcacctccaacaccaacatgtgcgccgtgattgacgggaacagcaccaccttcgagcaccagcagccgttgcaggaccggatgttcaaatttgaacttacccgccgtctggagcatgactttggcaaggtgacaaagcaggaagtcaaagagttcttccgctgggcgcaggatcacgtgaccgaggtggcgcatgagttccacgtcagaaagggtggagccaacaagagacccgcccccgatgacgctgataaaagcgagcccaagcgggcctgcccctcagtcgcagatccatcgacgtcagacgcggaaggagctccggtggactttgccgacaggtaccaaaacaaatgttctcgtcacgcgggcatgcttcagatgctgtttccctgcaagacatgcgagagaatgaatcagaatttcaacatttgcttcacgcacgggaccagggactgttcggagtgcttccccggcgtgtcagaatctcaaccggtcgtcagaaaaaggacgtatcggaaactctgtgccattcatcatctgctggggagggctcccgagattgcttgctcggcctgcgatctggtcaacgtggacctggatgactgtgtctctgagcaataaatgacttaaaccaggtatggctgccgatggttatcttccagattggctcgaggacaacctctctgagggcattcgcgagtggtgggacttgaaacctggagccccgaaacccaaagccaaccagcaaaagcaggacgacggccggggtctggtgcttcctggctacaagtacctcggacccttcaacggactcgacaagggggagcccgtcaacgcggcggacgcagcggccctcgagcacgacaaggcctacgaccagcagctcaaagcgggtgacaatccgtacctgcggtataaccacgccgacgccgagtttcaggagcgtctgcaagaagatacgtcttttgggggcaacctcgggcgagcagtcttccaggccaagaagcgggttctcgaacctctcggtctggttgaggaaggcgctaagacggctcctggaaagaagagaccggtagagcagtcaccccaagaaccagactcctcatcgggcatcggcaagacaggccagcagcccgctaaaaagagactcaattttggtcagactggcgacacagagtcagtccccgacccacaacctctcggagaacctccagcagccccctcaggtctgggacctaatacaatggcttcaggcggtggcgctccaatggcagacaataacgaaggcgccgacggagtgggtaattcctcgggaaattggcattgcgattccacatggctgggggacagagtcatcaccaccagcacccgaacctgggccctgcccacctacaacaaccacctctacaagcaaatctccaacggcacctcgggaggaagcaccaacgacaacacctactttggctacagcaccccctgggggtattttgacttcaacagattccactgccacttttcaccacgtgactggcagcgactcatcaacaacaattggggattccggcccaagagactcaacttcaagctcttcaacatccaggtcaaggaagtcacgacgaacgaaggcaccaagaccatcgccaataatctcaccagcaccgtgcaggtctttacggactcggagtaccagctaccgtacgtgctaggatcagctcaccagggatgtctgcctccgttcccggcggacgtcttcatggttcctcagtacggttatctaactctgaacaatggcagccaggccctgggacgttcctccttctactgcctggagtatttcccatcgcagatgctgagaaccggcaacaactttcagttcagctacaccttcgaggacgtgcctttccacagcagctacgcgcacagccaaagcctggacaggctgatgaatcccctcatcgaccagtacctgtattacctggtcagaacgcagacaaccgggactggagggacgcagactctggcattcagccaagcaggccctagctcaatggccagccaggctagaaactgggtgcccggaccgagctaccggcagcagcgcgtctccacgacaaccaaccagaacaacaacagcaactttgcctggacgggagctgccaaatttaaactgaacggccgagactctctaatgaaccccggcgtggccatggcttcacacaaggatgacgaggaccgcttcttcccttctagcggggtcctgattttcggcaagcaaggagccgggaatgatggagtggattacagccaagtgctgattacagatgaggaagaaatcaaggctaccaaccccgtggcaacagaggaatatggagcagtggccatcaacaaccaggccgctaatacgcaggcgcagaccggactcgtgcacaaccagggggtgattcccggcatggtgtggcagaacagagacgtgtacctgcagggtcccatctgggccaaaattcctcacacggacggcaactttcacccgtctcccctgatgggcggctttggactgaagcacccgcctcctcaaattctcatcaagaacacaccggttccagcggacccgccgcttaccttcaaccaggccaagctgaactctttcatcacgcagtacagcaccggacaggtcagcgtggaaatcgagtgggagctgcagaaagaaaacagcaaacgctggaatccagagattcagtacacttccaactactacaaatctacaaatgtggactttgctgtcaacacggaaggagtgtatagcgagcctcgccccattggcacccgttacctcacccgcaacctgtaattgcctgttaatcaataaaccggttaattcgtttcagtaaggaagaattcgtttaaa

SEQ ID NO: 3 - protein, 736 aa, Artificial Sequence
  synthetic
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPAAPSGLGPNTMASGGGAPMADNNEGADGVGNSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTTNEGTKTIANNLTSTVQVFTDSEYQLPYVLGSAHQGCLPPFPADVFMVPQYGYLTLNNGSQALGRSSFYCLEYFPSQMLRTGNNFQFSYTFEDVPFHSSYAHSQSLDRLMNPLIDQYLYYLVRTQTTGTGGTQTLAFSQAGPSSMANQARNWVPGPCYRQQRVSTTTNQNNNSNFAWTGAAKFKLNGRDSLMNPGVAMASHKDDEDRFFPSSGVLIFGKQGAGNDGVDYSQVLITDEEEIKATNPVATEEYGAVAINNQAANTQAQTGLVHNQGVIPGMVWQNRDVYLQGPIWAKIPHTDGNFHPSPLMGGFGLKHPPPQILIKNTPVPADPPLTFNQAKLNSFITQYSTGQVSVEIEWELQKENSKRWNPEIQYTSNYYKSTNVDFAVNTEGVYSEPRPIGTRYLTRNL

(19) United States
(12) Patent Application Publication
(10) Pub. No.: US 2023/0181763 A1
(43) Pub. Date: Jun. 15, 2023

(54) APPLICATION OF AAV44.9 VECTOR IN GENE THERAPY FOR THE INNER EAR

(71) Applicants: The United States of America, as represented by the Secretary, Department of Health and Human Sevice, Bethesda, MD (US); Consiglio Nazionale Delle Ricerche, Rome (IT)

(72) Inventors: John A. Chiorini, Dayton, MD (US); Giovanni Di Pasquale, Kensington, MD (US); Fabio Mammano, Monterotondo (IT); Veronica Zorzi, Monterotondo (IT)

(73) Assignees: The United States of America,as represented by the Secretary,Department of Health and Human Services, Bethesda, MD (US); Consiglio Nazionale Delle Ricerche, Rome (IT)

(21) Appl. No.: 17/922,300

(22) PCT Filed: Apr. 30, 2021
(86) PCT No.: PCT/US2021/030055

§ 371 Date: Oct. 28, 2022

Related U.S. Application Data
(60) Provisional application No. 63/018,140, filed on Apr. 30, 2020.

(51) Int. Cl. A61K 48/00; A61P 27/16; A61K 45/06

(52) U.S. Cl. CPC A61K 48/0041; A61P 27/16; A61K 45/06; A61K 48/0066

(57) ABSTRACT

Provided are methods of transducing hair cells of the inner ear in a subject comprising administering to the subject an adeno-associated viral (AAV) vector comprising a nucleic acid sequence encoding a capsid comprising the amino acid sequence of SEQ ID NO: 1, wherein the AAV vector further comprises a heterologous nucleic acid sequence. Additionally, methods of treating, preventing, or inhibiting a cochlear disorder or balance disorder in a subject comprising administering the AAV vector to the subject are provided.

### CROSS-REFERENCE TO RELATED APPLICATIONS

This patent application claims the benefit of U.S. Provisional Patent Application No. 63/018,140, filed on Apr. 30, 2020, which is incorporated by reference.

### STATEMENT REGARDING FEDERALLY SPONSORED RESEARCH OR DEVELOPMENT

This invention was made with Government support under project number ZIADE000695-14 by the National Institutes of Health, National Institute of Dental and Craniofacial Research. The Government has certain rights in the invention.

### SEQUENCE LISTING

Incorporated by reference in its entirety herein is a nucleotide/amino acid sequence listing submitted concurrently herewith.

### BACKGROUND OF THE INVENTION

Hearing and balance depend on the function of the inner ear sensory epithelium, which consists of sensory hair cells and a variety of supporting and epithelial cells that provide mechanical and trophic support for the hair cells. The development of efficient transgene delivery for the inner ear is an important step towards application of gene-based therapies for cochlear disorders.

### BRIEF SUMMARY OF THE INVENTION

The invention provides a method of transducing hair cells of the inner ear in a subject comprising administering to the subject an adeno-associated viral (AAV) vector comprising a nucleic acid sequence encoding a capsid comprising the amino acid sequence of SEQ ID NO: 1, wherein the AAV vector further comprises a heterologous nucleic acid sequence, thereby transducing hair cells of the inner ear of the subject. The hair cells to be transduced can be in any suitable portion of the inner ear, such as the organ of Corti, vestibular epithelia, utricular maculae, or saccular maculae of the subject.

Additionally, the invention provides a method of treating, preventing, or inhibiting a cochlear disorder or balance disorder in a subject comprising administering to the subject an AAV vector comprising a nucleic acid sequence encoding a capsid comprising the amino acid sequence of SEQ ID NO: 1, wherein the AAV vector further comprises a heterologous nucleic acid sequence, thereby treating, preventing, or inhibiting the cochlear disorder or balance disorder in the subject.

The cochlear disorder or balance disorder to be treated, prevented, or inhibited includes, but is not limited to, acute unilateral vestibulopathy (AUV), sudden sensorineural hearing loss (SSNHL), ototoxicity, benign paroxysmal positional vertigo (BPPV), tinnitus, Meniere's Disease, vertibular migraine, labyrinthitis, vestibular neuronitis, perilymph fistula, Mal de Debarquement syndrome (MdDS), and disorders caused by dysfunction of genes expressed in the hair cells of the inner ear.

The heterologous nucleic acid sequence comprised in the AAV vector can be operably linked to regulatory sequences which direct its expression in hairs of the inner ear and/or flanked by one or more inverted terminal repeat (ITR) sequences. The heterologous nucleic acid sequence can encode any human ortholog of the mouse hair cell-specific genes listed in Li et al., Sci. Data, 5: 180199 (2018)), including but not limited to ATOH1, BDNF, USH1, USH3, COCH, RERGL PIK3C2G, HSP70-1, KCNE1, KCNE2, AQP1-AQP4, OTOF, or SRRM4 (Nakano et al., PLOS Genetics, 8(10): e1002966 (2012)).

The capsid comprising the amino acid sequence of SEQ ID NO: 1 can be encoded by the nucleic acid sequence of SEQ ID NO: 2.

The AAV vector for use in the inventive methods can be comprised in a composition with a pharmaceutically acceptable carrier, wherein the composition can comprise one or more additional pharmaceutically active agents, such as corticosteroids, antibiotics, antivirals, and diuretics. Furthermore, the AAV vector (isolated or in a composition) can be administered concurrently or consecutively with one or more additional therapies (e.g., for cochlear disorders, such as corticosteroids, diuretics, low sodium diet, drug therapy, hearing aids, cochlear implants, and surgery, or balance disorders, such as antibiotics, corticosteroids, surgery, and vestibular rehabilitation therapy).

### BRIEF DESCRIPTION OF THE SEVERAL VIEWS OF THE DRAWINGS

FIGS. 1A and 1B are confocal immunofluorescence images showing cochlea cultured in vitro with AAV449 CMV-GFP. Cultures obtained from postnatal day 5 (P5) wild type C57BL6/N mouse pups were fixed and immunostained 72 hours after adding the viral vectors to the culture medium. Scale bar 50 μm. FIG. 1A shows culture transduced with AAV44.9 CMV-GFP, wherein only a few cells at the top of the image show expression of GFP. FIG. 1B is an image of putative Reissner's membrane cells at the extreme periphery of the culture, wherein again only a few cells show expression of GFP.

FIGS. 2A-2D are confocal immunofluorescence images of utricular macula transverse sections from wild type C57BL6/N mice transduced in vivo with AAV44.9 CMV-GFP. Mice were injected at P3 with AAV44.9 CMV-GFP and sacrificed 4 weeks later. (A) GFP staining showing hair cells, (B) actin staining, showing hair cell sterocilia, (C) DAPI staining, showing nuclei, and (D) combination. Virus transduction is evident in virtually all hair cells of utricular maculae. Scale bar: 20 μm.

FIGS. 3A-3D are confocal immunofluorescence images of the cochlear sensory epithelium (organ of Corti) from wild type C57BL6/N mice transduced in vivo with AAV44.9 CMV-GFP. Whole mount preparations from mice injected at P3 with AAV44.9 CMV-GFP and sacrificed 4 weeks later are shown. (A) GFP staining showing hair cells, (B) actin staining, (C) DAPI staining, and (D) combination. Magnification of hair cell region show that all inner hair cells are transduced whereas only a subset of the outer hair cells (asterisk) are transduced. Scale bar: 20 μm.

### DETAILED DESCRIPTION OF THE INVENTION

The ability of AAV vectors to transduce dividing and non-dividing cells, establish long-term transgene expression, and the lack of pathogenicity has made AAV vectors attractive for use in gene therapy applications. The lack of cross competition in binding experiments suggests that each AAV serotype has a distinct mechanism of cell entry.

AAV44.9 (corresponding to the amino acid sequence of SEQ ID NO: 1) and a modified AAV44.9 (with asparagine rather than serine at position 470 relative to SEQ ID NO: 1) were identified and described in U.S. Patent Application 2018/0355376, the disclosure of which is hereby incorporated by reference.

The amino acid sequence of capsid protein VP1 of AAV44.9 differs from the amino acid sequence of capsid protein VP1 of the most closely reported isolate AAVrh8R (GenBank Accession No. ACB55317) at several locations. In particular, the amino acid sequence of capsid protein VP1 of AAV44.9 (SEQ ID NO: 1, wherein residue 470 is serine) differs at positions 179, 473, and 483 relative to the amino acid sequence of capsid protein VP1 of the most closely reported isolate AAVrh8R (SEQ ID NO: 3). The modified AA44.9 (SEQ ID NO: 1, wherein residue 470 is asparagine) further differs at position 470 relative to the amino acid sequence of capsid protein VP1 of isolate AAVrh8R (SEQ ID NO: 3).

AAV44.9 was reported to have a high gene transfer activity in cell types including salivary gland cells, liver cells, and nerve cells (e.g., cells of the cortex, olfactory bulb, and brain stem and Purkinje cells of the cerebellum). However, as described herein, it was surprisingly discovered that AAV44.9 can transduce hair cells in the inner ear. This tropism was unexpected based on the results of in vitro primary cultures and was discovered only after in vivo delivery.

Transduction of Hair Cells of the Inner Ear

Therefore, the invention provides a method of transducing hair cells of the inner ear of a subject comprising administering to the subject an AAV vector (e.g., AAV44.9 vector) comprising a heterologous nucleic acid sequence. The AAV vector can comprise a nucleic acid sequence encoding a capsid comprising the amino acid sequence of SEQ ID NO: 1. The hair cells to be transduced can be in any suitable portion of the inner ear including, but not limited to, the organ of Corti, vestibular epithelia, utricular maculae, or saccular maculae of the subject.

The invention also provides method of treating or preventing a cochlear disorder or balance disorder in a subject comprising administering to the subject an AAV vector (e.g., AAV44.9 vector) comprising a heterologous nucleic acid sequence. The AAV vector can comprise a nucleic acid sequence encoding a capsid comprising the amino acid sequence of SEQ ID NO: 1.

The cochlear disorder or balance disorder to be treated, prevented, or inhibited can be any disorder of the inner ear including, but not limited to, acute unilateral vestibulopathy (AUV) (also known as vestibular neuritis), sudden sensorineural hearing loss (SSNHL) (also known as sudden deafness), ototoxicity (e.g., cisplatin-induced ototoxicity), benign paroxysmal positional vertigo (BPPV), tinnitus, Meniere's Disease, vertibular migraine, labyrinthitis, vestibular neuronitis, perilymph fistula, Mal de Debarquement syndrome (MdDS), and disorders caused by dysfunction of genes expressed in the hair cells of the inner ear.

The balance disorder can include symptoms, such as dizziness, vertigo, falling, staggering, lightheadedness, faintness, floating sensation, blurred vision, confusion, and/or disorientation.

Modifications of the VP1 Amino Acid Sequence of AAV44.9

In one embodiment, the AAV vector comprises a nucleic acid sequence encoding a capsid comprising the amino acid sequence of SEQ ID NO: 1, wherein residue 470 can be serine (AAV44.9) or serine (modified AAV44.9). The amino acid sequence of the VP1 capsid protein of AAV44.9 (SEQ ID NO 1, wherein residue is serine) can be modified, such as at one or more (e.g., one, two, or three) residues. For example, the VP1 capsid protein of AAV44.9 can be modified at residue 179 (T179S), residue 470 (S470N), residue 473 (S473N), and/or residue 483 (S383C) of SEQ ID NO: 1.

Alterations of the amino acid sequence to produce variant polypeptides can be done by a variety of means known to those skilled in the art. For instance, amino acid substitutions can be conveniently introduced into the polypeptides at the time of synthesis. Alternatively, site-specific mutations can be introduced by ligating into an expression vector a synthesized oligonucleotide comprising the modified site. Alternately, oligonucleotide-directed, site-specific mutagenesis procedures can be used. Direct gene synthesis of the mutant cDNA is also possible with current technology.

It is within the skill of the ordinary artisan to select synthetic and naturally-occurring amino acids that effect conservative or neutral substitutions for any particular naturally-occurring amino acids. The ordinarily skilled artisan desirably will consider the context in which any particular amino acid substitution is made, in addition to considering the hydrophobicity or polarity of the side-chain, the general size of the side chain and the pK value of side-chains with acidic or basic character under physiological conditions. For example, lysine, arginine, and histidine are often suitably substituted for each other, and more often arginine and histidine. As is known in the art, this is because all three amino acids have basic side chains, whereas the pK value for the side-chains of lysine and arginine are much closer to each other (about 10 and 12) than to histidine (about 6). Similarly, glycine, alanine, valine, leucine, and isoleucine are often suitably substituted for each other, with the proviso that glycine is frequently not suitably substituted for the other members of the group. This is because each of these amino acids are relatively hydrophobic when incorporated into a polypeptide, but glycine's lack of an α-carbon allows the phi and psi angles of rotation (around the α-carbon) so much conformational freedom that glycinyl residues can trigger changes in conformation or secondary structure that do not often occur when the other amino acids are substituted for each other. Other groups of amino acids frequently suitably substituted for each other include, but are not limited to, the group consisting of glutamic and aspartic acids; the group consisting of phenylalanine, tyrosine and tryptophan; and the group consisting of serine, threonine and, optionally, tyrosine. Additionally, the ordinarily skilled artisan can readily group synthetic amino acids with naturally-occurring amino acids.

The ordinarily skilled artisan can generate mutants or variants by, for example, substituting or mutating amino acids which are not critical for the function of the polypeptide. Ideally, mutations that do not modify the electronic or structural environment of the peptide are generated to retain optimal activity. For example, amino acid residues which are not responsible for folding or stability of the three-dimensional conformation of the polypeptide are candidate residues for mutation.

If desired, the polypeptide can be modified, for instance, by glycosylation, amidation, carboxylation, or phosphorylation, or by the creation of acid addition salts, amides, esters, in particular C-terminal esters, and N-acyl derivatives of the proteins of the invention. The polypeptide also can be modified to create protein derivatives by forming covalent or noncovalent complexes with other moieties in accordance with methods known in the art. Covalently-bound complexes can be prepared by linking the chemical moieties to functional groups on the side chains of amino acids comprising the proteins, or at the N- or C-terminus. Desirably, such modifications and conjugations do not adversely affect the activity of the polypeptide.

Polypeptides

The polypeptides (e.g., a polypeptide comprising a capsid comprising the amino acid sequence of SEQ ID NO: 1) can be prepared by any of a number of conventional techniques. In this respect, the polypeptide sequence can be synthetic, recombinant, isolated, and/or purified.

The polypeptide can be isolated or purified from a recombinant source. For instance, a DNA fragment encoding a desired polypeptide can be subcloned into an appropriate vector using well-known molecular genetic techniques. The fragment can be transcribed and the polypeptide subsequently translated in vitro. Commercially available kits also can be employed. The polymerase chain reaction optionally can be employed in the manipulation of nucleic acids.

The polypeptide also can be synthesized using an automated peptide synthesizer in accordance with methods known in the art. Alternately, the polypeptide can be synthesized using standard peptide synthesizing techniques well-known to those of skill in the art. In particular, the polypeptide can be synthesized using the procedure of solid-phase synthesis. If desired, this can be done using an automated peptide synthesizer. Removal of the t-butyloxycarbonyl (t-BOC) or 9-fluorenylmethyloxycarbonyl (Fmoc) amino acid blocking groups and separation of the polypeptide from the resin can be accomplished by, for example, acid treatment at reduced temperature. The protein-containing mixture then can be extracted, for instance, with diethyl ether, to remove non-peptidic organic compounds, and the synthesized polypeptide can be extracted from the resin powder (e.g., with about 25% w/v acetic acid). Following the synthesis of the polypeptide, further purification (e.g., using HPLC) optionally can be performed in order to eliminate any incomplete proteins, polypeptides, peptides or free amino acids. Amino acid and/or HPLC analysis can be performed on the synthesized polypeptide to validate its identity. For other applications according to the invention, it may be preferable to produce the polypeptide as part of a larger fusion protein, either by chemical conjugation or through genetic means, such as are known to those skilled in the art. In this regard, the invention also provides a fusion protein comprising the polypeptide and one or more other protein(s) having any desired properties or functions, such as to facilitate isolation, purification, analysis, or stability of the fusion protein.

Nucleic Acids

The invention also provides a nucleic acid encoding the polypeptide or a variant thereof. In one embodiment, the nucleic acid comprises, consists essentially of, or consists of the nucleic acid sequence of SEQ ID NO: 2 (which sequence comprises multiple DNA fragments isolated from AAV 44-9). “Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. It is generally preferred that the nucleic acid does not comprise any insertions, deletions, inversions, and/or substitutions. However, it may be suitable in some instances, as discussed herein, for the nucleic acid to comprise one or more insertions, deletions, inversions, and/or substitutions.

In an embodiment, the nucleic acid is recombinant. As used herein, the term “recombinant” refers to (i) molecules that are constructed outside living cells by joining natural or synthetic nucleic acid segments to nucleic acid molecules that can replicate in a living cell, or (ii) molecules that result from the replication of those described in (i) above. For purposes herein, the replication can be in vitro replication or in vivo replication.

The nucleic acid (e.g., DNA, RNA, cDNA, and the like) can be produced in any suitable matter including, but not limited to recombinant production and commercial synthesis. In this respect, the nucleic acid sequence can be synthetic, recombinant, isolated, and/or purified.

The nucleic acid can be constructed based on chemical synthesis and/or enzymatic ligation reactions using procedures known in the art. See, for example, Green et al. (eds.), Molecular Cloning, A Laboratory Manual, 4th Edition, Cold Spring Harbor Laboratory Press, New York (2012). For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N⁶-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Macromolecular Resources (Fort Collins, Colo.) and Synthegen (Houston, Tex.).

The nucleic acid encoding the polypeptide can be provided as part of a construct comprising the nucleic acid and elements that enable delivery of the nucleic acid to a cell, and/or expression of the nucleic acid in a cell. For example, the polynucleotide sequence encoding the polypeptide can be operatively linked to expression control sequences. An expression control sequence operatively linked to a coding sequence is ligated such that expression of the coding sequence is achieved under conditions compatible with the expression control sequences. The expression control sequences include, but are not limited to, appropriate promoters, enhancers, transcription terminators, a start codon (i.e., ATG) in front of a protein-encoding gene, splicing signal for introns, maintenance of the correct reading frame of that gene to permit proper translation of mRNA, and stop codons. Suitable promoters include, but are not limited to, a hVMD2 promoter, an SV40 early promoter, RSV promoter, adenovirus major late promoter, human CMV immediate early I promoter, poxvirus promoter, 30K promoter, 13 promoter, sE/L promoter, 7.5K promoter, 40K promoter, and C1 promoter.

A nucleic acid encoding the polypeptide can be cloned or amplified by in vitro methods, such as the polymerase chain reaction (PCR), the ligase chain reaction (LCR), the transcription-based amplification system (TAS), the self-sustained sequence replication system (3SR) and the Qβ replicase amplification system (QB). For example, a polynucleotide encoding the polypeptide can be isolated by polymerase chain reaction of cDNA using primers based on the DNA sequence of the molecule. A wide variety of cloning and in vitro amplification methodologies are well known to persons skilled in the art.

AAV Vectors

AAV is a member of the Parvoviridae family and comprises a linear, single-stranded DNA genome of less than about 5,000 nucleotides. AAV requires co-infection with a helper virus (i.e., an adenovirus or a herpes virus), or expression of helper genes, for efficient replication. AAV vectors used for administration of therapeutic nucleic acids typically have approximately 96% of the parental genome deleted, such that only the terminal repeats (ITRs), which contain recognition signals for DNA replication and packaging, remain. This eliminates immunologic or toxic side effects due to expression of viral genes. In addition, delivering specific AAV proteins to producing cells enables integration of the AAV vector comprising AAV ITRs into a specific region of the cellular genome, if desired (see, e.g., U.S. Pat. Nos. 6,342,390 and 6,821,511). Host cells comprising an integrated AAV genome show no change in cell growth or morphology (see, for example, U.S. Pat. No. 4,797,368).

The AAV ITRs flank the unique coding nucleotide sequences for the non-structural replication (Rep) proteins and the structural capsid (Cap) proteins (also known as virion proteins (VPs)). The terminal 145 nucleotides are self-complementary and are organized so that an energetically stable intramolecular duplex forming a T-shaped hairpin may be formed. These hairpin structures function as an origin for viral DNA replication by serving as primers for the cellular DNA polymerase complex. The Rep genes encode the Rep proteins Rep78, Rep68, Rep52, and Rep40. Rep78 and Rep68 are transcribed from the p5 promoter, and Rep 52 and Rep40 are transcribed from the p19 promoter. The Rep78 and Rep68 proteins are multifunctional DNA binding proteins that perform helicase and nickase functions during productive replication to allow for the resolution of AAV termini (see, e.g., Im et al., Cell, 61: 447-57 (1990)). These proteins also regulate transcription from endogenous AAV promoters and promoters within helper viruses (see, e.g., Pereira et al., J. Virol., 71: 1079-1088 (1997)). The other Rep proteins modify the function of Rep78 and Rep68. The cap genes encode the capsid proteins VP1, VP2, and VP3. The cap genes are transcribed from the p40 promoter.

The invention provides an AAV vector comprising a nucleic acid sequence encoding a capsid comprising the polypeptide (e.g., the polypeptide comprising, consisting essentially of, or consisting of the amino acid sequence of SEQ ID NO: 1), wherein the AAV vector further comprises a heterologous nucleic acid sequence. In one embodiment, the AAV vector comprises a nucleic acid sequence encoding a capsid comprising a VP1, VP2, and VP3 protein, wherein the VP1 protein comprises, consists essentially of, or consists of the amino acid sequence of SEQ ID NO: 1, and wherein the AAV further comprises a heterologous nucleic acid sequence.

A suitable recombinant AAV may be generated by culturing a packaging cell which contains a nucleic acid sequence encoding an AAV serotype capsid protein, or fragment thereof, as defined herein; a functional rep gene; any of the inventive vectors described herein; and sufficient helper functions to permit packaging of the inventive vector into the AAV capsid protein. The components required by the packaging cell to package the inventive AAV vector in an AAV capsid may be provided to the host cell in trans. Alternatively, any one or more of the required components (e.g., inventive vector, rep sequences, capsid sequences, and/or helper functions) may be provided by a stable packaging cell which has been engineered to contain one or more of the required components using methods known to those of skill in the art.

In an embodiment of the invention, the AAV vector is self-complementary. Self-complementary vectors may, advantageously, overcome the rate-limiting step of second-strand DNA synthesis and confer earlier onset and stronger gene expression.

In an embodiment of the invention, the AAV vector is a recombinant expression vector. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The AAV vectors (AAV44.9 vector or modified AAV44.9 vector) are not naturally-occurring as a whole. However, parts of the vectors can be naturally-occurring. The inventive recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring, non-naturally-occurring intemucleotide linkages, or both types of linkages. Preferably, the non-naturally occurring or altered nucleotides or intemucleotide linkages do not hinder the transcription or replication of the vector.

The AAV vector can be prepared using standard recombinant DNA techniques described in, for example, Green et al., supra.

In addition to the nucleic acid encoding the polypeptide, the AAV vector can comprise one or more nucleic acid sequences encoding one or more polypeptides for delivery and expression in a host (e.g., a mammal, such as a mouse, rat, guinea pig, hamster, cat, dog, rabbit, pig, cow, horse, or primate (e.g., human)).

The AAV vector can include one or more marker genes, which allow for selection of transformed or transfected hosts. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The AAV vector may further comprise regulatory sequences that permit one or more of the transcription, translation, and expression of nucleic acid comprised in the vector in a cell transfected with the vector or infected with a virus that comprises the vector. As used herein, “operably linked” sequences include both regulatory sequences that are contiguous with the nucleotide sequence and regulatory sequences that act in trans or at a distance to control the nucleotide sequence.

The regulatory sequences may include appropriate transcription initiation, termination, promoter, and enhancer sequences; RNA processing signals such as splicing and polyadenylation (polyA) signal sequences; sequences that stabilize cytoplasmic mRNA; sequences that enhance translation efficiency (i.e., Kozak consensus sequence); sequences that enhance protein stability; and when desired, sequences that enhance secretion of the encoded product. PolyA signal sequences may be synthetic or may be derived from many suitable species, including, for example, SV-40, human and bovine.

A large number of promoters, including constitutive, inducible, and repressible promoters, from a variety of different sources are well known in the art. Representative sources of promoters include for example, virus, mammal, insect, plant, yeast, and bacteria, and suitable promoters from these sources are readily available, or can be made synthetically, based on sequences publicly available, for example, from depositories such as the ATCC as well as other commercial or individual sources. Promoters can be unidirectional (i.e., initiate transcription in one direction) or bi-directional (i.e., initiate transcription in either a 3′ or 5′ direction). Non-limiting examples of promoters include, for example, the T7 bacterial expression system, pBAD (araA) bacterial expression system, the cytomegalovirus (CMV) promoter, the SV40 promoter, and the RSV promoter. Inducible promoters include, for example, the Tet system, the Ecdysone inducible system, the T-REX™ system (Invitrogen, Carlsbad, Calif.), LACSWITCH™ System (Stratagene, San Diego, Calif.), and the Cre-ERT tamoxifen inducible recombinase system.

The term “enhancer” as used herein, refers to a DNA sequence that increases transcription of, for example, a nucleic acid sequence to which it is operably linked. Enhancers can be located many kilobases away from the coding region of the nucleic acid sequence and can mediate the binding of regulatory factors, patterns of DNA methylation, or changes in DNA structure. A large number of enhancers from a variety of different sources are well known in the art and are available as or within cloned polynucleotides (from, e.g., depositories such as the ATCC as well as other commercial or individual sources). A number of polynucleotides comprising promoters (such as the commonly-used CMV promoter) also comprise enhancer sequences. Enhancers can be located upstream, within, or downstream of coding sequences. For example, the nucleic acid encoding the polypeptide can be operably linked to a CMV enhancer/chicken β-actin promoter (also referred to as a “CAG promoter”).

When the AAV vector is for administration to a host (e.g., human), the vector preferably has a low replicative efficiency in a target cell (e.g., no more than about 1 progeny per cell or, more preferably, no more than 0.1 progeny per cell are produced). Replication efficiency can readily be determined empirically by determining the virus titer after infection of the target cell.

Heterologous Nucleic Acid Sequence

In one embodiment, the invention provides an AAV vector comprising a nucleic acid sequence encoding a capsid comprising the polypeptide (e.g., the amino acid sequence of SEQ ID NO: 1), wherein the AAV vector further comprises a heterologous nucleic acid sequence. A heterologous nucleic acid sequence refers to a nucleic acid sequence that is heterologous to the vector sequences flanking the heterologous nucleic acid sequence. The heterologous nucleic acid sequence can encode a polypeptide, protein, or other product of interest. The heterologous nucleic acid sequence is operatively linked to regulatory components in a manner which permits transcription, translation, and/or expression in a host cell.

The heterologous nucleic acid sequence can include a reporter sequence, which upon expression produces a detectable signal. Such reporter sequences include, without limitation, nucleic acid sequences encoding β-lactamase, β-galactosidase (LacZ), alkaline phosphatase, thymidine kinase, green fluorescent protein (GFP), chloramphenicol acetyltransferase (CAT), luciferase, membrane bound proteins including, for example, CD2, CD4, CD8, the influenza hemagglutinin protein, and others well known in the art to which high affinity antibodies directed thereto exist or can be produced by conventional means, and fusion proteins comprising a membrane bound protein appropriately fused to an antigen tag domain from, for example, hemagglutinin or Myc.

These coding sequences, when associated with regulatory elements which drive their expression, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry. For example, when the sequence is the LacZ gene, the presence of the vector carrying the signal is detected by assays for beta-galactosidase activity. When the sequence encodes green fluorescent protein or luciferase, the vector carrying the signal may be measured visually by color or light production in a luminometer.

The heterologous nucleic acid sequence also can be a non-reporter sequence encoding a product which is useful in biology and medicine, such as proteins, peptides, RNA, enzymes, dominant negative mutants, or catalytic RNAs. Desirable RNA molecules include tRNA, dsRNA, ribosomal RNA, catalytic RNAs, siRNA, small hairpin RNA, trans-splicing RNA, and antisense RNAs. One example of a useful RNA sequence is a sequence which inhibits or extinguishes expression of a targeted nucleic acid sequence in the treated host. Typically, suitable target sequences include oncologic targets and viral diseases.

The heterologous nucleic acid sequence can be used to correct or ameliorate gene deficiencies, which may include deficiencies in which normal genes are expressed at less than normal levels or deficiencies in which the functional gene product is not expressed.

Suitable heterologous nucleic acid sequences may be readily selected by one of skill in the art. The selection of the heterologous nucleic acid sequence is not considered to be a limitation of this invention.

In one embodiment, the heterologous nucleic acid sequence can be a nucleic acid sequence encoding gene products involved in cochlear disorders or balance disorders including, but not limited to, any human ortholog of the mouse hair cell-specific genes listed in Li et al., Sci. Data, 5: 180199 (2018)), including but not limited to ATOH1, BDNF, USH1, USH3, COCH, RERGL PIK3C2G, HSP70-1, KCNE1, KCNE2, AQP1-AQP4, OTOF, or SRRM4 (Nakano et al., PLOS Genetics, 8(10): e1002966 (2012)).

The heterologous nucleic acid sequence can be flanked by one or more inverted terminal repeat (ITR) sequences.

The AAV vector can comprise multiple (two, three, four, five, six, seven, eight, nine, or ten) heterologous nucleic acid sequences. Multiple heterologous nucleic acid sequences can be used, for example, to correct or ameliorate a gene defect caused by a multi-subunit protein. In certain situations, a different heterologous nucleic acid sequence may be used to encode each subunit of a protein, or to encode different peptides or proteins. This is desirable when the size of the nucleic acid encoding the protein subunit is large, e.g., for an immunoglobulin, the platelet-derived growth factor, or a dystrophin protein. In order for the cell to produce the multi-subunit protein, a cell is infected with the recombinant virus containing each of the different subunits. Alternatively, different subunits of a protein may be encoded by the same nucleic acid sequence. In this case, a single heterologous nucleic acid sequence includes the nucleic acid encoding each of the subunits, with the nucleic acid for each subunit separated by an internal ribozyme entry site (IRES). This is desirable when the size of the nucleic acid encoding each of the subunits is small, e.g., the total size of the nucleic acid encoding the subunits and the IRES is less than five kilobases. As an alternative to an IRES, the nucleic acid may be separated by sequences encoding a 2A peptide, which self-cleaves in a post-translational event. This 2A peptide is significantly smaller than an IRES, making it well suited for use when space is a limiting factor. More often, when the heterologous nucleic acid sequence is large, consists of multi-subunits, or two heterologous nucleic acid sequences are co-delivered, rAAV carrying the desired heterologous nucleic acid sequence(s) or subunits are co-administered to allow them to concatamerize in vivo to form a single vector genome. In such an embodiment, a first AAV vector may carry an expression cassette which expresses a single heterologous nucleic acid sequence and a second AAV vector may carry an expression cassette which expresses a different heterologous nucleic acid sequence for co-expression in the host cell. However, the selected heterologous nucleic acid sequence may encode any biologically active product or other product, e.g., a product desirable for study.

Compositions

The polypeptide, nucleic acid, or AAV vector can be formulated as a composition (e.g., pharmaceutical composition) comprising the polypeptide, nucleic acid, or AAV vector and a carrier (e.g., a pharmaceutically or physiologically acceptable carrier). Furthermore, the polypeptide, nucleic acid, AAV vector, or composition can be used in the methods described herein alone or as part of a pharmaceutical formulation.

The composition (e.g., pharmaceutical composition) can comprise more than one polypeptide, nucleic acid, AAV vector, or composition of the invention. Alternatively, or in addition, the composition can comprise one or more (e.g., one, two, three, or more) additional pharmaceutically active agents or drugs, such as corticosteroids, antibiotics, antivirals, and diuretics.

The carrier can be any of those conventionally used and is limited only by physio-chemical considerations, such as solubility and lack of reactivity with the active compound(s) and by the route of administration. The pharmaceutically acceptable carriers described herein, for example, vehicles, adjuvants, excipients, and diluents, are well-known to those skilled in the art and are readily available to the public. It is preferred that the pharmaceutically acceptable carrier be one which is chemically inert to the active agent(s) and one which has no detrimental side effects or toxicity under the conditions of use.

The choice of carrier will be determined in part by the particular polypeptide, nucleic acid, vector, or composition thereof of the invention and other active agents or drugs used, as well as by the particular method used to administer the polypeptide, nucleic acid, AAV vector, or composition thereof.

The composition also can be formulated to enhance transduction efficiency. In addition, a person of ordinary skill in the art will appreciate that the one or more of the polypeptides, nucleic acids, or AAV vectors can be present in a composition with other therapeutic or biologically-active agents. For example, factors that control inflammation, such as ibuprofen or steroids, can be part of the composition to reduce swelling and inflammation associated with in vivo administration of one or more of the polypeptides, nucleic acids, or AAV vectors. Antibiotics, i.e., microbicides and fungicides, can be present to treat existing infection and/or reduce the risk of future infection, such as infection associated with gene transfer procedures.

Administration Routes and Dosing

The polypeptide, nucleic acid, AAV vector, or composition thereof can be administered to the subject by any method. For example, the polypeptide, nucleic acid encoding the polypeptide, or AAV vector can be introduced into a cell (e.g., in a subject) by any of various techniques, such as by contacting the cell with the nucleic acid or the AAV vector as part of a construct, as described herein, that enables the delivery and expression of the nucleic acid. Specific protocols for introducing and expressing nucleic acids in cells are known in the art.

Any suitable dose of the polypeptide, nucleic acid, AAV vector, or composition thereof can be administered to a subject. The appropriate dose will vary depending upon such factors as the subject's age, weight, height, sex, general medical condition, previous medical history, and disease progression, and can be determined by a clinician. The amount or dose should be sufficient to effect the desired biological response, e.g., a therapeutic or prophylactic response, in the subject over a clinically reasonable time frame.

For example, the polypeptide can be administered in a dose of about 0.05 mg to about 10 mg (e.g., 0.1 mg, 0.5 mg, 1 mg, 2 mg, 3 mg, 4 mg, 5 mg, 6 mg, 7 mg, 8 mg, 9 mg, and ranges thereof) per vaccination of the host (e.g., mammal, such as a human), and preferably about 0.1 mg to about 5 mg per vaccination. Several doses (e.g., 1, 2, 3, 4, 5, 6, or more) can be provided (e.g., over a period of weeks or months).

The dosing period may be appropriately determined depending on the therapeutic progress. In embodiments, the dosing period may comprise less than one year, less than 9 months, less than 8 months, less than 7 months, less than 6 months, less than 5 months, less than 4 months, less than 3 months, less than 2 months, or one month. In other embodiments, the dosing period may comprise three doses per day, two doses per day, or one dose per day for the length of the dosing period.

A suitable dose of the AAV vector can include about 1×10⁵ to about 1×10¹² (e.g., 1×10⁶, 1×10⁷, 1×10⁸, 1×10⁹, 1×10¹⁰, 1×10¹¹, and ranges thereof) plaque forming units (pfus), although a lower or higher dose can be administered to a host.

The polypeptide, nucleic acid, AAV vector, or composition thereof can be administered to the subject by various routes including, but not limited to, topical, subcutaneous, intramuscular, intradermal, intraperitoneal, intrathecal, intravenous, subretinal injection, and intravitreal injection.

In one embodiment, the polypeptide, nucleic acid, AAV vector, or composition can be directly administered (e.g., locally administered) by direct injection into the ear or by topical application. When administering the AAV vector into the inner ear, a hole can be made in the round window membrane or a hole can be drilled in the otic capsule bone and then the AAV vector is injected.

When multiple administrations are given, the administrations can be at one or more sites in a subject and a single dose can be administered by dividing the single dose into equal portions for administration at one, two, three, four or more sites on the individual.

The polypeptide, nucleic acid, AAV vector, or composition thereof is useful for preventing emergence of cochlear disorders, arresting progression of cochlear disorders, or eliminating cochlear disorders. More particularly, the polypeptide, nucleic acid, AAV vector, or composition thereof can be used to prevent, inhibit, or delay the development of cochlear disorders in an individual. The polypeptide, nucleic acid, AAV vector, or composition thereof can also be used to ameliorate at least one symptom of the cochlear disorders, such as by preventing, inhibiting, reversing, or delaying progression of the cochlear disorder (e.g., hearing loss) in the individual.

The polypeptide, nucleic acid, AAV vector, or composition thereof can be administered concurrently or consecutively with one or more (e.g., one, two three, or more) additional therapies. For example, the one or more additional therapies can be for cochlear disorders including, but not limited to, corticosteroids, diuretics, low sodium diet, drug therapy, hearing aids, cochlear implants, surgery, and combinations thereof. Alternatively or additionally, the one or more additional therapies can be for balance disorders including, but not limited to, antibiotics, corticosteroids, surgery, vestibular rehabilitation therapy, and combinations thereof.

The following example further illustrates the invention but, of course, should not be construed as in any way limiting its scope.

### Example

This example demonstrates that AAV44.9 can transduce hair cells of the inner ear in vivo.

First, an attempt was made to transduce cochlear organotypic cultures with AAV44.9 expressing CMV driven GFP (AAV44.9 CMV-GFP). Cochleae were dissected form postnatal day 5 (P5) mouse pups (where PO is day of birth) in ice-cold Hepes buffered Hanks' balanced salt solutions (HBSS) and placed onto glass coverslips coated with Cell Tak. Cultures were incubated in Dulbecco's modified Eagle's medium supplemented with the F12 growth factor (DMEM/F12) supplemented with 5% fetal bovine serum (FBS) and maintained at 37° C. for 1 day.

Transduction with viral constructs was attempted by adding vector (100 μL; 3×10¹²) in culture medium devoid of FBS. Cultures were kept in this medium at 37° C. for the first 24 hours to favor viral transduction and thereafter maintained in DMEM/F12 supplemented with FBS up to 48 hours.

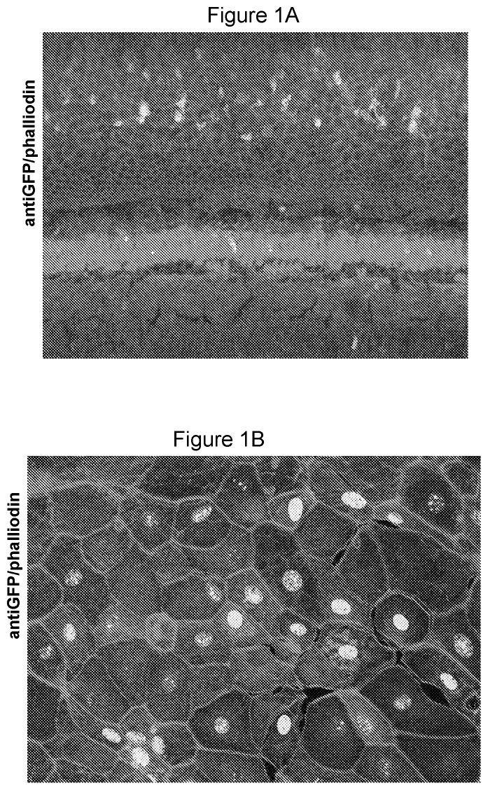

As shown in FIGS. 1A and 1B, virus transduction was only minimally observed in culture in vitro.

Based on the in vitro data, one would not expect that the AAV44.9 would be able to successfully transfect hair cells in vivo. However, the inventors surprisingly discovered that virtually all hair cells were transduced by AAV44.9 in vivo as described in the following experiments.

Canalostomy

P3 pups of wild type C57BL6/N mice were anaesthetized with xylazine 0.45 μg/g and zolazepam 0.15 μg/g diluted in physiological solution. After induction of anesthesia, mice were placed under a dissection microscope and the semicircular canal was exposed by a dorsal post-auricular approach.

Viral solution diluted in DMEM/F12 was injected into the endolymphatic space (500 nl injected volume, 3 nl/s injection speed) with a micropump—controlled micro syringe equipped with a 36 G needle. For every transduction experiment, —10¹⁰ viral particles were injected. Ten minutes after injection, the needle was slowly removed.

Immunohistochemistry

Mice that were injected with viral solution were sacrificed four weeks after injection and the entire bony labyrinth (inner ear) comprising cochlea, saccule, utricle, and the three semiciruclar canals was extracted bilaterally. Samples were fixed in 4% paraformaldehyde and decalcified in EDTA (0.3 M). Decalcified specimens were included in 3% agarose dissolved in PBS and cut in 100 μm thickness steps using a vibratome. Tissue slices were permeabilized with 0.1% Triton X-100, dissolved in BSA 2% solution. A rabbit polyclonal GFP selective antibody was used to distinguish the exogenous fusion proteins from endogenous proteins. Secondary antibody was applied for 1 hour at room temperature while F-Actin was stained by incubation with AlexaFluor 568 phalloidin and nuclei were stained with DAPI. Samples were mounted onto glass slides with a mounting medium and analyzed using a confocal microscope equipped with an oil-immersion objective.

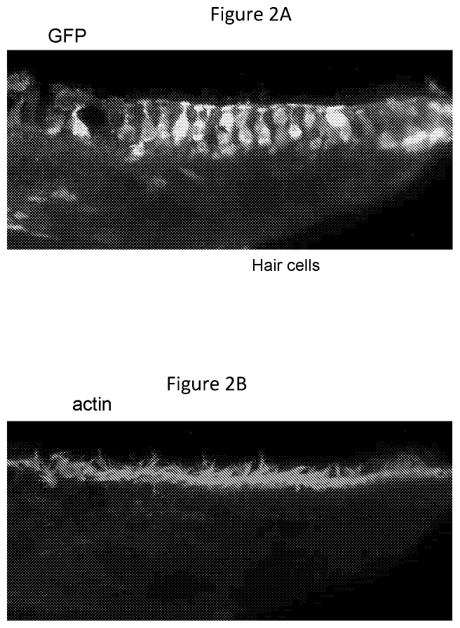

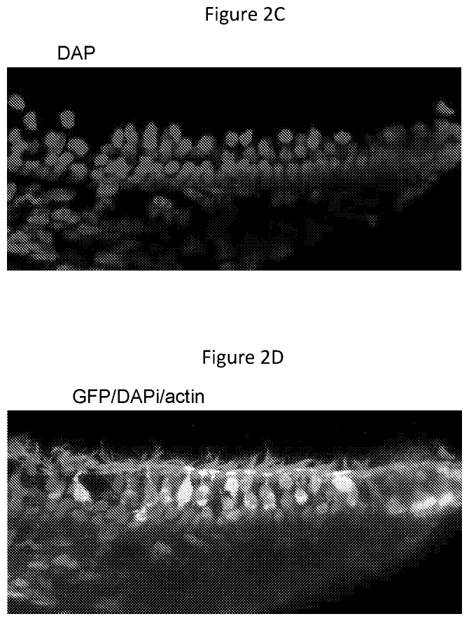

As shown in FIGS. 2A-2D, AAV44.9 CMV-GFP virus transduction was observed in virtually all hair cells of utricular maculae.

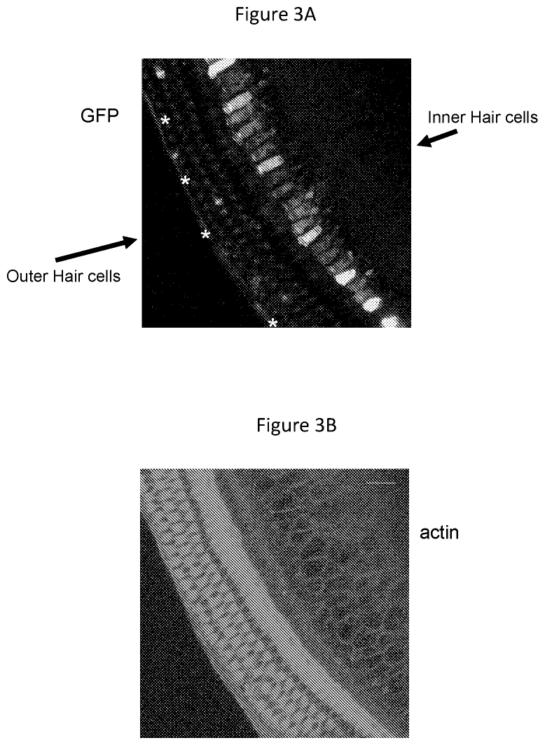

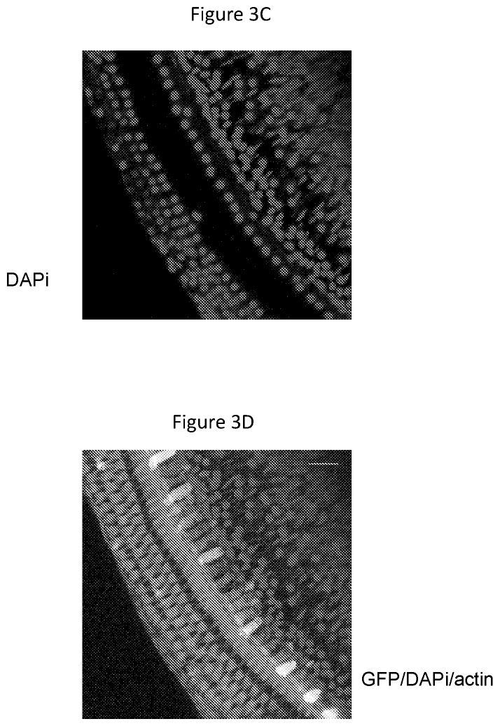

Similarly, GFP expression was observed in inner hair cells in the organ of Corti of injected mice four weeks after delivery of AAV44.9 CMV-GFP viral vector at postnatal day 3 (P3). FIGS. 3A-3D are a magnification of the hair cell region showing that all hair cells in the organ of Corti were transduced whereas only a subset of the outer hair cells were transduced.

These results demonstrate that AAV44.9 can successfully transduce hair cells, particularly inner hair cells, of the inner ear in vivo.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of transducing hair cells of the inner ear in a subject comprising administering of the subject an adeno-associated viral (AAV) vector comprising a nucleic acid sequence encoding a capsid comprising the amino acid sequence of SEQ ID NO: 1, wherein the AAV vector further comprises a heterologous nucleic acid sequence, thereby transducing hair cells of the inner ear of the subject.

2. The method of claim 1 wherein the hair cells are in the organ of Corti, vestibular epithelia, utricular maculae, or saccular maculae of the subject.

3. A method of treating, preventing, or inhibiting a cochlear disorder or balance disorder in a subject comprising administering to the subject an AAV vector comprising a nuclei acid sequence encoding a capsid comprising the amino acid sequence of SEQ ID NO:1, wherein the AAV vector further comprises a heterologous nucleic acid sequence, thereby treating, preventing, or inhibiting the cochlear disorder in the subject.

4. The method of claim 3, wherein the cochlear disorder or balance disorder is selected from the group consisting of acute unilateral vestibulopathy (AUV), sudden sensorineural hearing loss (SSNHL), ototoxicity, benign paroxysmal positional vertigo (BPPV), tinnitus, Meniere's Disease, vertibular-migraine, labyrinthitis, vestibular neuronitis, perilymph fistula, Mal de Debarquement syndrome (MdDS), and a disorder caused by dysfunction of a gene expressed in the hair cells of the inner ear.

5. The method of claim 3 wherein, the AAV vector is administered concurrently or consecutively with one or more additional therapies for cochlear disorders or balance disorders.

6. The method of claim 5, wherein the one or more additional therapies for cochlear disorders or balance disorders is selected from the group consisting of antibiotics, corticosteroids, diuretics, low sodium diet, drug therapy, hearing aids, cochlear implants, vestibular rehabilitation therapy, and combinations thereof.

7. The method of claim 1, wherein the heterologous nucleic acid sequence is operably linked to regulatory sequences which direct expression of the heterologous nucleic acid sequence in hairs of the inner ear.

8. The method of claim 1, wherein the heterologous nucleic acid sequence encodes ATOH1, BDNF, USH1, USH3, COCH, RERGL PIK3C2G, HSP70-1, KCNE1, KCNE2, AQP1-AQP4, SRRM4, or OTOF.

9. The method of claim 1, wherein the heterologous nucleic acid sequence is flanked by one or more inverted terminal repeat (ITR) sequences.

10. The method of claim 1, wherein the capsid is encoded by the nucleic acid sequence of SEQ ID NO: 2.

11. The method of claim 1, wherein the AAV vector is in a composition with a pharmaceutically acceptable carrier.

12. The method of claim 11, wherein the composition further comprises one or more additional pharmaceutically active agents.

13. The method of claim 12, wherein the one or more additional pharmaceutically active agents is selected from the group consisting of corticosteroids, antibiotics, antivirals, diuretics, and combinations thereof.

14. The method of claim 1, wherein residue 470 of the amino acid sequence of SEQ ID NO: 1 is serine.

15. The method of claim 1, wherein residue 470 of the amino acid sequence of SEQ ID NO: 1 is asparagine.
